(19) United States
(12) Patent Application Publication
(10) Pub. No.: US 2022/0202700 A1
(43) Pub. Date: Jun. 30, 2022

(54) COMPOSITIONS FOR THE TREATMENT OF VIRAL PULMONARY INFECTIONS

(71) Applicant: Paul Strohecker, Greenbelt, MD (US)

(72) Inventor: Paul Strohecker, Greenbelt, MD (US)

(21) Appl. No.: 17/695,489

(22) Filed: Mar. 15, 2022

Related U.S. Application Data
(63) Continuation of application No. 16/946,572, filed on Jun. 26, 2020.

(51) Int. Cl. A61K 9/00; A61K 47/14; A61K 31/133; A61K 47/18

(52) U.S. Cl. CPC A61K 9/008; A61K 9/0078; A61K 9/0075; A61K 31/133; A61K 47/186; A61K 47/14

(57) ABSTRACT

The disclosure relates to a method for treating a viral pulmonary infection (e.g., COVID-19 infection), the method comprising the step of delivering a composition comprising at least one bronchodilator or a pharmaceutically acceptable salt thereof and at least one pulmonary surfactant or a pharmaceutically acceptable salt thereof to the airway of a subject in need of treatment for the pulmonary viral infection.

### CROSS-REFERENCE TO RELATED APPLICATION

This application is a continuation of U.S. patent application Ser. No. 16/946,572, filed Jun. 26, 2020, which application and publication is incorporated herein by reference in its entirety.

### BACKGROUND

Three coronaviruses have crossed the species barrier to cause deadly pneumonia in humans since the beginning of the 21st century: severe acute respiratory syndrome coronavirus (SARS-CoV), Middle-East respiratory syndrome coronavirus, and SARS-CoV-2. SARS-CoV emerged in the Guangdong province of China in 2002 and spread to five continents through air travel routes, infecting 8,098 people and causing 774 deaths. In 2012, MERS-CoV emerged in the Arabian Peninsula, where it remains a major public health concern, and was exported to 27 countries, infecting a total of 2,494 individuals and claiming 858 lives. A previously unknown coronavirus, named SARS-CoV-2, was discovered in December 2019 in Wuhan, Hubei province of China and was sequenced and isolated by January 2020. SARSCoV-2 is associated with an ongoing outbreak of atypical pneumonia (Covid-2019) that has affected over 8.5 million people and killed more than 450,000 of those affected in >60 countries as of Jun. 19, 2020.

On Jan. 30, 2020, the World Health Organization declared the SARS-CoV-2 epidemic a public health emergency of international concern. MERS-CoV was suggested to originate from bats, but the reservoir host fueling spillover to humans is unequivocally dromedary camels. Both SARS-CoV and SARS-CoV-2 are closely related and originated in bats, who most likely serve as reservoir host for these two viruses. Whereas palm civets and racoon dogs have been recognized as intermediate hosts for zoonotic transmission of SARS-CoV between bats and humans, the SARS-CoV-2 intermediate host remains unknown. The recurrent spillovers of coronaviruses in humans along with detection of numerous coronaviruses in bats, including many SARS-related coronaviruses (SARSr-CoVs), suggest that future zoonotic transmission events may continue. In addition to the highly pathogenic zoonotic pathogens SARS-CoV, MERS-CoV, and SARS-CoV-2, all belonging to the b-coronavirus genus, four low-pathogenicity coronaviruses are endemic in humans: HCoV-0043, HCoVHKU1, HCoV-NL63, and HCoV-229E.

### SUMMARY

To date, no therapeutics or vaccines are approved against any human-infecting coronaviruses. Coronavirus entry into host cells is mediated by the transmembrane spike (S) glycoprotein that forms homotrimers protruding from the viral surface. S comprises two functional subunits responsible for binding to the host cell receptor (S1 subunit; contains the receptor-binding domain (RBD)) and fusion of the viral and cellular membranes (82 subunit). For many CoVs, S is cleaved at the boundary between the S1 and S2 subunits, which remain non-covalently bound in the prefusion conformation. The distal S1 subunit comprises the receptor-binding domain(s) and contributes to stabilization of the prefusion state of the membrane-anchored S2 subunit that contains the fusion machinery. For all CoVs, S is further cleaved by host proteases at the so-called S2 site located immediately upstream of the fusion peptide. This cleavage has been proposed to activate the protein for membrane fusion via extensive irreversible conformational changes. As a result, coronavirus entry into susceptible cells is a complex process that requires the concerted action of receptor-binding and proteolytic processing of the S protein to promote virus-cell fusion.

Different coronaviruses use distinct domains within the S1 subunit to recognize a variety of attachment and entry receptors, depending on the viral species. Endemic human coronaviruses OC43 and HKU1 attach via their S domain A (SA) to 5-Nacetyl-9-O-acetyl-sialosides found on glycoproteins and glycolipids at the host cell surface to enable entry into susceptible cells. MERS-CoV S, however, uses domain A to recognize non-acetylated sialoside attachment receptors, which likely promote subsequent binding of domain B (SB) to the entry receptor, dipeptidyl-peptidase 4. SARS-CoV and several SARS-related coronaviruses (SARSr-CoV) interact directly with angiotensin-converting enzyme 2 (ACE 2) via SB to enter target cells.

As the coronavirus S glycoprotein is surface-exposed and mediates entry into host cells, it is the focus of the therapeutic approach described herein. But those of skill in the art will recognize that, even though the current description is framed in the context of treating COVID-19, the disclosure is more general than that. The disclosure also encompasses a method for treating a pulmonary infection caused by an enveloped virus, of which the novel corona virus is but one example, the method comprising the step of delivering a composition comprising at least one bronchodilator or a pharmaceutically acceptable salt thereof and at least one pulmonary surfactant or a pharmaceutically acceptable salt thereof to the airway of a subject in need of treatment for the infection. In addition to the viruses described herein, examples of enveloped viruses (e.g., enveloped respiratory viruses) also include, but are not limited to, influenza viruses, viruses of the Mononegavirales order, including those in the Orthomyxoviridae family and Paramyxoviridae family. Those of skill in the art will appreciate that the Orthomyxoviridae family includes the influenza viruses and the Paramyxoviridae family includes the parainfluenza viruses (PIVs), human respiratory syncytial virus (RSV), and human metapneumovirus (hMPV). See Clin Microbiol Rev. 21: 274-290 (2008).

### DESCRIPTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Pore formation, and antecedent membrane convergence, are facilitated by the SPIKE-like membrane proteins. Interaction of these membrane proteins allows for the comingling of parasite and host phospholipid sections of the membranes. In the case of influenza A and model MDCK cells, this convergence is inhibited by both 1-palmitoyl-2-oleoyl-sn-glycero-3-phosphoglycerol (POPG) and phosphatidyl inositol (PI):

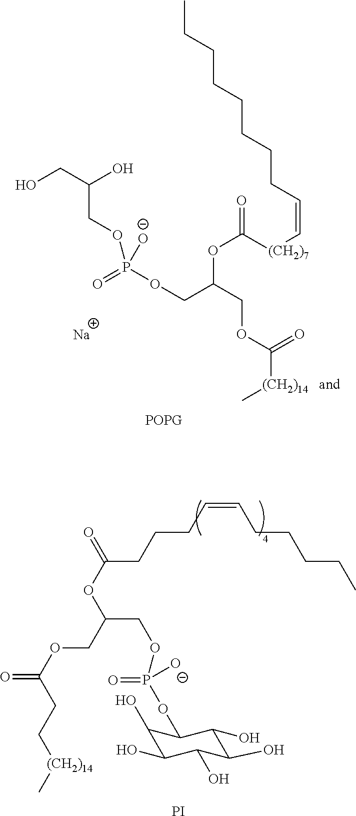

POPG and PI are pulmonary surfactants having a high binding affinity for the influenza A virus. POPG and PI prevent the binding of the influenza A virus to the surrogate host cells. Because of the congeneric relationship between the novel coronavirus, COVID-19, and influenza A, this same phenomenon could be seen in COVID-19 infections, where POPG and PI could preferentially bind to the novel coronavirus, rendering the virus unavailable for infecting pulmonary cells. In this scenario, POPG and PI would not act as viral envelope disruptors, but instead as viral sequesters. The net result is inhibition, and therefore reduction, of propagation of an established COVID-19 infection.

Regarding reduction of pulmonary inflammation, POPG has been shown to lower total inflammatory cell infiltrates (neutrophils and lymphocytes) by >90%. Virus-induced proinflammatory cytokines were reduced by both POPG and PI. Similar action of POPG and PI on COVID-19 infections, thereby complementing the virus-reducing properties of POPG and PI in ameliorating the disease and potentially reducing mortality from COVID-19.

It has been shown that the overall enveloped virus infection-reducing behavior of POPG and PI—both anionic pulmonary surfactants—is due to their functioning as deactivators of Toll-like receptors (TLR); they suppress innate immunity. By strict physico-chemical analogy, this TLR deactivation should be seen with coronavirus in COVID-19 cases.

Albuterol, a short-acting beta-2 agonist bronchodilator, in inhaler form, is being used in COVID-19 treatment. Albuterol has been shown to be an effective bronchodilator that does not deleteriously affect pulmonary surfactant levels. POPG has been shown to be viricidal in mice and ferrets. In addition, POPG has been shown to have anti-inflammation properties in the respiratory system of mice.

One goal of the instant disclosure is, therefore, the combination of the therapeutic effects of compositions comprising bronchodilators (e.g., non-steroidal bronchodilators), for example, beta-2 agonists like albuterol; and pulmonary surfactants, like the phosphoglycerol (PG)-type pulmonary surfactant, POPG, for the treatment of COVID-19. PG-type pulmonary surfactants include compounds of the formula:

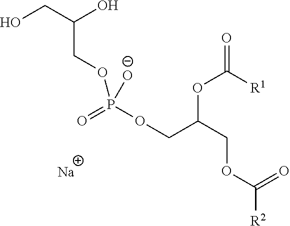

wherein R¹ and R² are each independently an alkyl group.

The term “alkyl” as used herein refers to substituted or unsubstituted straight chain and branched alkyl groups and cycloalkyl groups having from 1 to 40 carbon atoms (C₁-C₄₀), 1 to about 20 carbon atoms (C₁-C₂₀₎, 1 to 12 carbons (C₁-C₁₂), 1 to 8 carbon atoms (C₁-C₈), or from 10 to 20 carbon atoms (C₁₀-C₂₀). The alkyl groups can comprise one, two, three, four, five, six or more unsaturated portions (e.g., a —CH═CH— group, wherein the —CH═CH— group can be E or Z). Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with, for example, amino, hydroxy, cyano, carboxy, nitro, thio, alkoxy, and halogen (e.g., F, Cl or Br) groups.

Non-PG-type pulmonary surfactants are also contemplated herein. PC-type (phosphatidylcholine) pulmonary surfactants are also contemplated herein:

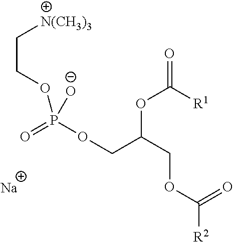

wherein R¹ and R² are defined herein, such as phosphatidylcholine:

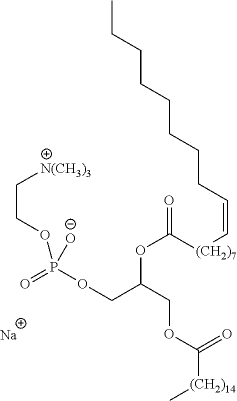

Another example of a PC-type pulmonary surfactant is dipalmitoylphosphatidylcholine (DPPC).

The combination of at least one bronchodilator and a PG-type surfactant will significantly reduce viral concentration and reduce inflammation in the COVID-19 disease, while simultaneously increasing pulmonary surface area and oxygen exchange effects exerted by the bronchodilator.

More generally, the COVID-19 treatment possibility presented herein is the combination of at least one bronchodilator or pharmaceutically acceptable salts thereof with at least one pulmonary surfactant or pharmaceutically acceptable salts thereof. An example of a specific combination contemplated herein is albuterol (e.g., as free base or sulphate) and POPG, but formulations are not limited to this combination. For example, a pulmonary surfactant, such as POPG, could be combined with other bronchodilators including, but not limited to, e.g., salmeterol (e.g., as xinafoate), ephedrine, adrenaline, fenoterol (e.g., as hydrobromide), formoterol (e.g., as fumarate), isoprenaline, metaproterenol, phenylephrine, phenylpropanolamine, pirbuterol (e.g., as acetate), reproterol (e.g., as hydrochloride), rimiterol, terbutaline (e.g., as sulphate), isoetharine, tulobuterol, 4-hydroxy-7-[2-[[2-[[3-(2-phenylethoxy)propyl]sulfonyl]ethyl]amino]ethyl-2(3H)-benzothiazolone, aclidinium, arformoterol, glycopyrrolate, indacaterol, levalbuterol, olodaterol, revefenacin, tiotropium, umeclidinium, and pharmaceutically acceptable salts thereof.

The compositions described herein can also contain additional components (e.g., solvents, excipients, and the like), including components that can enhance the effectiveness of the compositions described herein in the treatment for treating a pulmonary infection caused by an enveloped virus, including the novel corona virus, the method comprising the step of delivering a composition comprising at least one bronchodilator or a pharmaceutically acceptable salt thereof and at least one pulmonary surfactant or a pharmaceutically acceptable salt thereof. For example, antiviral drug combinations have been shown to have enhanced activity for important human respiratory viruses. Rimantadine or amantadine combined with ribavirin shows increased antiviral effects in vitro and in experimental animal models. See, e.g., Antiviral Research 29: 45-48 (1996), which is incorporated by reference as if fully set forth herein. Accordingly, in addition to the at least one bronchodilator or a pharmaceutically acceptable salt thereof and at least one pulmonary surfactant or a pharmaceutically acceptable salt thereof, the compositions described herein can further comprise additional antiviral drugs, or combinations thereof, including, for example, rimantadine or amantadine combined with ribavirin.

As used herein, the term “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

Alternatively, any of the foregoing bronchodilators can be combined with any suitable pulmonary surfactant including, but not limited to, modified natural pulmonary surfactants such as bovine lipid pulmonary surfactant (BLES™, BLES Biochemicals, Inc. London, Ont), calfactant (INFASURF™, Forest Pharmaceuticals, St. Louis, Mo.), bovactant (ALVEOFACT™, Thomae, Germany), bovine pulmonary surfactant (PULMONARY SURFACTANT TA™, Tokyo Tanabe, Japan), poractant alfa (CUROSURF™, Chiesi Farmaceutici SpA, Parma, Italy), and beractant (SURVANTA™, Abbott Laboratories, Inc., Abbott Park, 111). Other examples of pulmonary surfactants include, but are not limited to, reconstituted surfactants, such as the compositions disclosed in EP 2 152 288, WO2008/011559, WO2013/120058, all of which are incorporated herein by reference in their entireties. Examples of reconstituted surfactants include lucinactant (SURFAXIN™, Windtree-Discovery Laboratories Inc., Warrington, Pa.) and compositions comprising 1.5% of SP-C33(leu) acetate; 0.2% of Mini-B(leu) acetate; and DPPC:POPG in a 50:50 weight ratio. See, e.g., Table 2 of Example 2 of WO2010/139442, which is incorporated herein by reference in its entirety.

Additional examples of pulmonary surfactants include modified natural pulmonary surfactants. Examples of modified natural pulmonary surfactants include poractant alfa (CUROSURF™).

The dose of the pulmonary surfactant to be administered will vary with, among other things, the type of pulmonary surfactant, as well as the weight and age of the subject receiving the compositions described herein, which comprise a bronchodilator and a pulmonary surfactant. Although those of skill in the relevant art will be readily able to determine these factors and to adjust the dosage accordingly a dose of the pulmonary surfactant could be from about 0.1 mg/kg to about 200 mg/kg, such as 100 mg/kg to about 200 mg/kg.

The dose of bronchodilator will also vary with, among other things, the type of bronchodilator, as well as the weight and age of the subject receiving the compositions described herein, which comprise a bronchodilator and a pulmonary surfactant. Although those of skill in the relevant art will be readily able to determine these factors and to adjust the dosage accordingly a dose of the bronchodilator could be from about 0.05 mg/kg to about 10 mg/kg, such as 0.1 mg/kg to about 5 mg/kg

A general aspect of the current disclosure is the delivery of the compositions described herein, which comprise a bronchodilator and a pulmonary surfactant, to the airway of a subject (e.g., the lower airways, including the anatomic regions below the larynx including the trachea and lungs, e.g., a human subject). The delivery can be accomplished using any suitable device that accomplishes the dosing. For pulmonary administration, the compositions described herein can be delivered in a particle size effective for reaching the airways of a subject (e.g., the lower airways).

Suitable devices for delivering the compositions described herein include devices that can deliver small particles, e.g., less than about 10 microns, about 3 to about 5 microns or particles with small stokes radius. For example, the compositions described herein can be delivered via inhalation by any of a variety of inhalation devices known in the art for administration of a therapeutic agent by inhalation. These devices capable of depositing aerosolized formulations in the airways of a patient include but are not limited to metered dose inhalers, sprayers, nebulizers, and dry powder generators. All such devices can dispense the compositions described herein in an aerosol form. Such aerosols can be comprised of nanoparticles, microparticles, solutions (both aqueous and nonaqueous), or solid particles.

Nebulizers, including AERx Aradigm, the Ultravent nebulizer (Mallinckrodt), and the Acorn II nebulizer (Marquest Medical Products) produce aerosols from solutions.

Metered dose inhalers such as e.g., the Ventolin metered dose inhaler, typically use a propellant gas and require actuation during inspiration.

Suitable dry powder inhalers like Turbuhaler (Astra), Rotahaler (Glaxo), Diskus (Glaxo), Spiros inhaler (Dura/Elan), devices, and the Spinhaler powder inhaler (Fisons), use breath-actuation of a mixed powder. Metered dose inhalers, dry powder inhalers, and the like generate small particle aerosols.

These specific examples of commercially available inhalation devices are intended to be a representative of specific devices suitable for the delivery of the compositions described herein, and are not intended as limiting the scope of the disclosure.

The compositions described herein can be administered as a topical spray or powder to the airways of a subject (e.g., a mammal, such as a human subject) by a delivery device (e.g., oral or nasal inhaler, aerosol generator, oral dry powder inhaler, through a fiberoptic scope, or via syringe during surgical intervention). These numerous drug delivery devices capable of drug distribution to the airways can use a liquid, semisolid, and solid composition. The site of deposition in the airways and the deposition area depend on several parameters related to the delivery device, such as mode of administration, particle size of the formulation, and velocity of the delivered particles. Several in vitro and in vivo methods that may be used by one of ordinary skill in the art to study distribution and clearance of therapeutics delivered to the airways, all of which is incorporated in its entirety, herein. Thus, any of these devices may be selected for use in the instant disclosure, given one or more advantages for a particular indication, technique, and subject. These delivery devices include but are not limited to devices producing aerosols (metered-dose inhalers (MDIs)), nebulizers and other metered and nonmetered inhalers.

In general, current container-closure system designs for inhalation spray drug products include both pre-metered and device-metered presentations using mechanical or power assistance and/or energy from patient inspiration for production of the spray plume. Pre-metered presentations may contain previously measured doses or a dose fraction in some type of units (e.g., single, multiple blisters, or other cavities) that are subsequently inserted into the device during manufacture or by the patient before use. Typical device-metered units have a reservoir containing formulation sufficient for multiple doses that are delivered as metered sprays by the device itself when activated by the patient.

In one example, delivery devices are contemplated that are able to distribute the compositions described herein expressly to the mucosa of the lower airways in a subject in need of such treatment. For example, the delivery device is able to distribute the composition expressly to the mucosa of the lower airways in a subject in need of such treatment, with a small amount of composition reaching the pharynx and upper airways. In another example, the delivery device is able to distribute the compositions described herein expressly to the mucosa of the lower airways in a subject in need of such treatment, with a minimal amount distributed to the posterior pharynx and the upper airways. In still another example, the delivery device is able to distribute the composition expressly to the mucosa of the lower airways in a subject in need of such treatment, with a negligible amount distributed to the posterior pharynx and the upper airways.

The instant disclosure also incorporates multidose metering or non-metering inhalers that are especially suited for repeated administrations and can provide numerous doses (e.g., 60 to up to about 130 doses, or more) either with or without stabilizers and preservatives.

Administration of the compositions described herein as a spray can be produced by forcing a suspension or solution of the compositions through a nozzle under pressure. The nozzle size and configuration, the applied pressure, and the liquid feed rate can be chosen to achieve the desired output and particle size to optimize deposition expressly in the lower airways. An electrospray can be produced, for example, by an electric field in connection with a capillary or nozzle feed. Particles of compositions described herein delivered by a sprayer can have a particle size less than about 20 microns, in the range below 10 microns, and from about 3 to 5 microns, but other particle sizes may be appropriate depending on the device, composition, and subject needs.

Commercially available nebulizers for liquid formulations, including jet nebulizers and ultrasonic nebulizers, can also be useful for administration to the airways. Liquid formulations can be directly nebulized and lyophilized powder nebulized after reconstitution. Alternatively, the compositions described herein can be aerosolized using a metered dose inhaler, or inhaled as a lyophilized and milled powder. In addition, the compositions described herein can be instilled through a bronchoscope, placed directly into the affected regions.

The compositions described herein can also be administered by a metered dose inhaler. The metered-dose inhaler (MDI) can contain therapeutically active ingredients dissolved or suspended in a propellant, a mixture of propellants, or a mixture of solvents, propellants, and/or other excipients in compact pressurized aerosol dispensers. The MDI may discharge up to several hundred metered doses of the composition. Depending on the composition, each actuation may contain from a few micrograms (μg) up to milligrams (mg) of the active ingredients delivered in a volume typically between 25 and 100 microliters. In a MDI, a propellant, at least one bronchodilator and at least one pulmonary surfactant, and various excipients or other compounds are contained in a canister as a mixture including a liquified compressed gas (propellants). Actuation of the metering valve releases the mixture as an aerosol, e.g., containing particles in the size range of less than about 20 μm, particles in a size range of less than about 10 μm, and particles in a size range of below about 5 μm. The desired aerosol particle size can be obtained by various methods known to those of skill in the art, including jet-milling, spray drying, critical point condensation, or other methods well known to one of ordinary skill in the art.

The compositions described herein for use with a metered-dose inhaler device can include a finely divided powder containing the at least one bronchodilator and the at least one pulmonary surfactant as a suspension in a non-aqueous medium, for example, suspended in a propellant with the aid of a surfactant or solubilizing agent. The propellant can be any conventional material including but not limited to chlorofluorocarbon, a hydrochlorofluorocarbon, a hydrofluorocarbon, or a hydrocarbon, including trichlorofluoromethane, dichlorodifluoromethane, dichlorotetrafluoroethanol and 1,1,1,2-tetrafluoroethane, HFA-134a (hydrofluroalkane-134a), HFA-227 (hydrofluroalkane-227). Hydrofluorocarbon is a preferred propellant. An additional surfactant can be chosen to stabilize the at least one bronchodilator as a suspension in the propellant, to protect the active agent against chemical degradation. In some cases, solution aerosols are preferred using solvents such as ethanol for more water-soluble bronchodilators. Additional agents including a protein can also be included in the composition.

One of ordinary skill in the art will recognize that the methods of the current invention can be achieved by administration to the airways (e.g., the lower airways) of at least one bronchodilator and at least one pulmonary surfactant via devices not described herein. The disclosure also incorporates unit-dose metering and non-metering spray devices that are especially suited for single administration. These devices are typically used for acute short-term treatments and single-dose delivery and can accommodate a liquid, powder, or mixture of both formulations of the composition. However, in certain circumstances, these unit dose devices may be preferred over multidose devices when used repeatedly in a particular way. Such uses may include but are not limited to repeated procedures where a sterile device is preferred.

Another embodiment of the invention provides for a single-dose syringe prefilled with the composition appropriate for treating COVID-19 in the airways (e.g., lower airways) of the subject. The prefilled syringe may be sterile or nonsterile and used in dose administration during procedures to a subject in need of COVID-19 therapy. An example of an application where a syringe is preferred includes, but is not limited to, the distribution of composition through an endoscope. These examples are not intended to be limiting and one skilled in the art will appreciate that other options exist for delivery of the composition expressly to the airways and these are incorporated herein.

The compositions described herein, comprising at least one bronchodilator and at least one pulmonary surfactant, can directly administered to the lower airways. Such administration may be carried out via use of an intrapulmonary aerolizer, which create an aerosol containing the composition and which may be directly installed into the lower airways. Exemplary aerolizers are described in U.S. Pat. Nos. 5,579,578; 6,041,775; 6,029,657; 6,016,800, and 5,594,987 all of which are herein incorporated by reference in their entirety. Such aerolizers are small enough in size so they can be inserted directly into the lower airways, for example into an endotracheal tube or even into the trachea. In one embodiment, the aerolizer may be positioned near the carina, or first bifurcation, of the lung. In another embodiment, the aerolizer is positioned so as to target a specific area of the lung, for example an individual bronchus, bronchiole, or lobe. Since the spray of the device is directly introduced into the lungs, losses due to deposition of the aerosol due to deposition on the walls of the nasal passages, mouth, throat, and trachea are avoided. Optimally, the droplet size produced by such suitable aerolizer is somewhat larger than those produced by ultrasonic nebulizers. Therefore, the droplets are less likely to be exhaled and thus leading to a delivery efficiency of virtually 100%. In addition, the delivery of the compositions has a highly uniform pattern of distribution.

An intrapulmonary aerosolizer can also be used to deliver the compositions described herein to the airways of a subject. An intrapulmonary aerosolizer comprises an aerosolizer attached to a pressure generator for delivery of liquid as an aerosol and which can be positioned in close proximity to the lungs by being inserted into the trachea directly or into an endotracheal tube or bronchoscope positioned within the trachea. Such an aerolizer may operate at pressures of up to about 2000 psi and produces particles with a medium particle size of 12 μm.

An intrapulmonary aerosolizer can comprise a substantially elongated sleeve member, a substantially elongated insert, and a substantially elongated body member. The sleeve member includes a threaded inner surface, which is adapted to receive the insert, which is a correspondingly threaded member. The threaded insert provides a substantially helical channel. The body member includes a cavity on its first end, which terminates by an end wall at its second end. The end wall includes an orifice extending therethrough. The body member is connected with the sleeve member to provide the aerosolizer of the present invention. The aerosolizer is sized to accommodate insertion into the trachea of a subject for administration of the compositions described herein. For operation of the device, the aerosolizer is connected by a suitable tube with a liquid pressure driver apparatus. The liquid pressure driver apparatus is adapted to pass liquid material (e.g., a composition comprising at least one bronchodilator and at least one pulmonary surfactant) therefrom which is sprayed from the aerosolizer. Due to the location of the device deep within the trachea, the liquid material is sprayed in close proximity to the lungs, with resulting improved penetration and distribution of the sprayed material in the lungs.

Alternatively, such an aerosolizer, sized for intratracheal insertion, is adapted for spraying a composition containing one or more proinflammatory cytokine inhibitors directly into the airways (e.g., in the lower airways). The aerosolizer is placed into connection with a liquid pressure driver apparatus for delivering of the liquid composition. The aerosolizer comprises a generally elongated sleeve member, which defines a first end and a second end and includes a longitudinally extending opening therethrough. The first end of the sleeve member is placed in connection with the liquid pressure driver apparatus. A generally elongated insert is also provided. The generally elongated insert defines a first end and a second end and is received within at least a portion of the longitudinally extending opening of the sleeve member. The insert includes an outer surface which has at least one substantially helical channel provided surrounding its outer surface which extends from the first end to the second end. The substantially helical channel of the insert is adapted to pass the liquid material, which is received by the sleeve member. A generally elongated body member is also included which is in connection with the sleeve member. The body member includes a cavity provided in its first end, which terminates at an end wall which is adjacent its second end. The end wall is provided having an orifice therein for spraying the liquid material, which is received from the insert. The portions of the sleeve member, insert and body member, in combination, are of sufficient size to allow for intratracheal insertion. A method of using such an aerosolizer includes the steps of connecting an aerosolizer with a first end of a hollow tube member and connecting the second end of the hollow tube member with the liquid pressure driver apparatus. The method further includes the steps of providing the aerosolizer in the trachea or into a member which is provided in the trachea, and then activating the liquid pressure driver apparatus for spraying a composition containing one or more proinflammatory cytokine inhibitors therefrom.

Alternatively, a powder dose composition comprising at least one bronchodilator and at least one pulmonary surfactant is directly administered to the lower airways via use of a powder dispenser. Examples of powder dispensers are disclosed in U.S. Pat. Nos. 5,513,630, 5,570,686 and 5,542,412, all of which are herein incorporated in their entirety. Such a powder dispenser is adapted to be brought into connection with an actuator, which introduces an amount of a gas for dispensing the powder dose. The dispenser includes a chamber for receiving the powder dose and a valve for permitting passage of the powder dose only when the actuator introduces the gas into the dispenser. The powder dose is passed from the dispenser via a tube to the lower airways of the subject. The powder dose may be delivered intratracheally, near the carina, which bypasses the potential for large losses of the powder dose to e.g., the mouth, throat, and trachea. In addition, in operation the gas passed from the actuator serves to slightly insufflate the lungs, which provides increased powder penetration. For the intratracheal insertion, the tube can be effected through an endotracheal tube in anesthetized, ventilated subjects, including animal or human patients, or in conscious subjects, the tube be inserted directly into the trachea preferably using a small dose of local anesthetic to the throat and/or a small amount of anesthetic on the tip of the tube, in order to minimize a “gag” response.

Alternatively, a composition comprising at least one bronchodilator and at least one pulmonary surfactant is directly administered to the lower airways. Such administration may be carried out via use of an aerolizer, which create an aerosol containing the composition and which may be directly installed into the lower airways. Examples of aerolizers are disclosed in U.S. Pat. Nos. 5,579,758; 6,041,775; 6,029,657; 6,016,800; 5,606,789; and 5,594,987 all of which are herein incorporated by reference in their entirety. The disclosure thus provides for the methods of administering compositions comprising at least one bronchodilator and at least one pulmonary surfactant directly to the lower airways by an aerolizer.

The compositions described herein can also be delivered using an “intratracheal aerosolizer” device which methodology involving the generation of a fine aerosol at the tip of a long, relatively thin tube that is suitable for insertion into the trachea. Thus, the present invention provides a new method of use for this aerosolizer technology in a microcatheter as adapted herein, for use in the lower airways in the prevention, treatment, and care of COVID-19.

An aerosolizing microcatheter can be used to administer a composition comprising at least one bronchodilator and at least one pulmonary surfactant. Examples of such catheters and their use, termed “intratracheal aerosolization” which involves the generation of a fine aerosol at the tip of a long, relatively thin tube that is suitable for insertion into the trachea, are disclosed in U.S. Pat. Nos. 5,579,758; 5,594,987; 5,606,789; 6,016,800; and 6,041,775.

Additional methods for delivering the compositions described herein include delivery using a microcatheter aerosolizer device (U.S. Pat. Nos. 6,016,800 and 6,029,657) adapted for nasal and paranasal sinus delivery and uses to deliver the compositions comprising at least one bronchodilator and at least one pulmonary surfactant in the treatment of COVID-19. One advantage of this microcatheter aerosolizer is the potential small size (0.014″ in diameter), and thus capable of being easily inserted into the working channel of a human flexible (1 to 2 mm in diameter) or ridged endoscope and thereby directed partially or completely into the ostium of a paranasal sinus.

One of ordinary skill in the art will recognize that the methods of the current disclosure can be achieved by administration of a composition described herein comprising at least one bronchodilator and at least one pulmonary surfactant via devices not described herein.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading can occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

In the methods described herein, the steps can be carried out in any order without departing from the principles of the invention, except when a temporal or operational sequence is explicitly recited. Furthermore, specified steps can be carried out concurrently unless explicit claim language recites that they be carried out separately. For example, a claimed step of doing X and a claimed step of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more.

The term “substantially no” as used herein refers to less than about 30%, 25%, 20%, 15%, 10%, 5%, 3%, 2%, 1%, 0.5%, 0.1%, 0.05%, 0.001%, or at less than about 0.0005% or less or about 0% or 0%.

Those skilled in the art will appreciate that many modifications to the embodiments described herein are possible without departing from the spirit and scope of the present disclosure. Thus, the description is not intended and should not be construed to be limited to the examples given but should be granted the full breadth of protection afforded by the appended claims and equivalents thereto. In addition, it is possible to use some of the features of the present disclosure without the corresponding use of other features. Accordingly, the foregoing description of or illustrative embodiments is provided for the purpose of illustrating the principles of the present disclosure and not in limitation thereof and can include modification thereto and permutations thereof.

### EXAMPLES

The disclosure can be better understood by reference to the following examples which are offered by way of illustration. The disclosure is not limited to the examples given herein.

An example of a composition comprising at least one bronchodilator and at least one pulmonary surfactant is shown in Table 1.

TABLE 1

| Component | % w/w | CAS # |
|---|---|---|
| POPG/PI | 1.3 | 185435-28-3 |
| Albuterol Sulfate | 0.1 | 51022-70-9 |
| Normal Saline | 0.9 | 7647-14-5. |

What is claimed is:

1. A method for treating pulmonary inflammation comprising administering a pharmaceutical composition to the airway of a subject in need thereof, wherein the composition consists of:
albuterol or a pharmaceutically acceptable salt thereof;
1-palmitoyl-2-oleoyl-sn-glycero-3-phosphoglycerol (POPG) or a pharmaceutically acceptable salt thereof; and
at least one of a solvent, a propellant, and an excipient;
wherein:
the albuterol and the POPG are the sole therapeutic agents.

2. The method of claim 1, wherein the pulmonary inflammation is caused by a coronavirus.

3. The method of claim 2, wherein the pulmonary inflammation is caused by SARS-CoV-2.

4. The method of claim 1, wherein the airway is the upper airway.

5. The method of claim 1, wherein the airway is the lower airway.

6. The method of claim 1, wherein the airway is the lungs.

7. The method of claim 1, wherein the composition is an aerosolized composition.

8. The method of claim 1, wherein the composition is administered with an inhalation device.

9. The method of claim 1, wherein the composition is administered with a dry powder inhaler (DPI),metered dose inhaler (MDI), a sprayer, a nebulizer, or a dry powder generator.

10. The method of claim 1, wherein the albuterol is albuterol sulfate.

11. The method of claim 1, wherein the excipient is a solubilizing agent or a suspension stabilizer.

12. The method of claim 1, wherein the solvent comprises ethanol.

13. The method of claim 1, wherein the excipient is an additional surfactant.

14. The method of claim 1, wherein the propellant comprises at least one of a chlorofluorocarbon, a hydrochlorofluorocarbon or a hydrofluorocarbon.

15. The method of claim 1, wherein the propellant comprises at least one of trichlorofluoromethane, dichlorodifluoromethane, dichlorotetrafluoroethanol 1,1,1,2-tetrafluoroethane, FIFA-134a (hydrofluroalkane-134a), and FIFA-227 (hydrofluroalkane-227).

16. The method of claim 1, wherein the composition is administered to the airway of the subject as an aerosol.

17. The method of claim 16, wherein the aerosol comprises particles having a size of less than about 10 μm.

18. The method of claim 1, wherein the composition is a solution or a suspension.
